(19) United States
(12) Patent Application Publication
(10) Pub. No.: US 2023/0404848 A1
(43) Pub. Date: Dec. 21, 2023

(54) CLINOCHLORE COMPOSITIONS AND METHODS OF USE

(71) Applicant: Gina Wu, Sedona, AZ (US)

(72) Inventor: Gina Wu, Sedona, AZ (US)

(21) Appl. No.: 18/206,504

(22) Filed: Jun. 6, 2023

Related U.S. Application Data
(60) Provisional application No. 63/350,260, filed on Jun. 8, 2022.

(51) Int. Cl. A61H 39/00; A61K 41/00

(52) U.S. Cl. CPC A61H 39/007; A61K 41/0004

(57) ABSTRACT

A composition that includes at least one clinochlore and a resin is described. A method of treating a user that includes applying a composition including a piezoelectric material such as a clinochlore and a polymerized resin to at least one acupuncture or acupressure point of a user is also described.

### CROSS-REFERENCE TO RELATED APPLICATIONS

The present application relies on the disclosure of and claims priority to and the benefit of the filing date of U.S. Provisional Patent Application No. 63/350,260, filed Jun. 8, 2022, which is hereby incorporated by reference in its entirety.

### FIELD

The present disclosure relates generally to compositions for therapeutic purpose. The present disclosure relates particularly to minerals that create a piezo-electric effect when encased in a contracting/compressing resin formed into disks and other shapes, that can then be used for therapeutic purposes.

### BACKGROUND

Traditional Chinese medical theory describes twelve major meridians that connect specific organs or networks of organs in a human body, creating a channel system of communication throughout the body. Each meridian begins at a person's fingertips or toes, connects to the brain, and then connects to an organ associated with that certain meridian. Vital energy, a life force called qi (ch'i), flows through these meridians. When one of these meridians is blocked or the energy flow out of balance, illness can occur.

Certain points lie along the meridians that can be treated with acupuncture needles or acupressure to unblock or balance the energy flow through the meridians. When these points are stimulated, it can help relax or strengthen a person. The acupuncture and acupressure treatments are provided by a practitioner skilled in such treatments, who inserts needles temporarily into or massages these select points.

It would be desirable to provide a way to provide stimulate the energy flow in the body without the need to insert needles or need a practitioner to constantly apply pressure.

### SUMMARY

This disclosure is a composition of crushed clinochlore mineral encased in a resin that compresses or contracts as it hardens creating a voltage potential or electric charge. The composition is formed into a hard disk and the disk is placed over an acupressure or acupuncture point on a person's body and secured to the skin with medical grade removable adhesive tape.

The disclosure features a composition that includes at least one clinochlore and a resin. The disclosure also features a method of treating a user that includes applying a composition including a piezoelectric material such as a clinochlore and a polymerized resin to at least one acupuncture or acupressure point of a user.

Features of the composition and method can include the following. The at least one clinochlore can be seraphinite, and also include colerainite, kämmerite, pennine, penninite, prochlorite, ripidolite, and/or sheridanite. The resin can be a plant resin. The plant resin can be amber. The resin can be synthetic. The synthetic resin can be acrylic, epoxy, polyester, phenolic, alkyd, polycarbonate, polyamide, polyurethane, silicone, polyethylene, polystyrene, or polypropylene. The composition can be formed into a disk or bead. The disk can have a diameter in the range of 3 mm to 40 mm and a height in the range of 2 mm to 10 mm. The at least one clinochlore can include clinochlore fragments. The clinochlore fragments can be piezoelectric, such that the polymerized resin mechanically stresses the clinochlore fragments to produce a voltage potential. The method can include applying the composition to the at least one acupuncture or acupressure point of a user through adhesive tape.

It should be understood that the composition and method are not to be considered limitations on the invention defined by the claims. The featured composition and method can be implemented in one or more ways using one or more features depicted in the drawings, described in the detailed description, and set forth in the claims.

### BRIEF DESCRIPTION OF THE DRAWINGS

The accompanying drawings illustrate certain aspects and principles of the implementations set forth, and should not be construed as limiting.

FIG. 1 is a side view of a composite disk in loaf form according to one implementation.

FIG. 2 is a side view of another composite disk in loaf form according to one implementation.

FIG. 3 is a top perspective view of a composite disk in cylindrical form according to one implementation.

FIG. 4 is a top perspective view of another composite disk in cylindrical form according to one implementation.

FIG. 5 is a top perspective view of a composite disk in dome form according to one implementation.

FIG. 6 is a top perspective view of another composite disk in dome form according to one implementation.

FIG. 7 is a black and white photograph of various sizes and shapes of composite disks according to various implementations.

FIG. 8 is a schematic illustration of foot with a composite disk taped to the skin of the foot according to one implementation.

### DETAILED DESCRIPTION

Reference will now be made in detail to various illustrative implementations. It is to be understood that the following discussion of the implementations is not intended to be limiting.

This disclosure is directed to a composition of crushed clinochlore mineral or other piezoelectric material encased in a hardened resin, and methods of using the composition in treating a user in Traditional Chinese medicine. According to one implementation, the composition is formed into a hard disk and the disk is placed over an acupressure or acupuncture point on a person's body and secured to the skin with medical grade removable adhesive tape.

Clinochlore is a mineral in the chlorite group having the chemical formula (Mg,Fe²⁺)₅Al(Si₃Al)O₁₀(OH)₈. Clinochlore comes in a variety of colors including green, yellowish green, olive green, blackish green, bluish green, white, and pink. Certain natural formations of clinochlore are green and have inclusions that look like angel wings. These formations are known as seraphinite (also known as chlorite jade), named after the Seraphim, the angels known in various religions. Other varieties of clinochlore include colerainite, kämmerite, pennine, penninite, prochlorite, ripidolite, and sheridanite. According to some implementations, seraphinite is the sole clinochlore mineral in the composition, while in other implementations seraphinite is included in combination with other types of clinochlore in the composition.

Seraphinite is available commercially as raw material, in naturally shaped crystals or polished beads, and originates in the Korshunovskoye iron skarn deposit in the Irkutskaya Oblast of Eastern Siberia. Mineralogical characteristics of seraphinite are described in the literature (see Manqu Zhao, Yan Lan, Na Yu, and Tianyang Zhang (2006): Study on Mineralogical Characteristics of Chlorite Jade from Russia. Journal of Gems & Gemmology 8(2), 14-16, incorporated by reference in its entirety). Zhao et al. reported for a particular sample that “the chlorite jade is in dark green, semi-translucent to opaque, and lepidoblastic texture, with hardness of 2.5, refractive index 1.58 and density 2.60 g/cm³”. This seraphinite raw material is crushed to form a collection of mineral flakes and residual granular fragments, which increases the surface area of the mineral in relation to the final form. The flakes and fragments can have a length or width of 10 microns to 1 cm, including 15 microns, 20 microns, 25 microns, 50 microns, 75 microns, 100 microns 150 microns, 200 microns, 250 microns, 300 microns, 400 microns, 500 microns, 600 microns 700 microns, 800 microns, 900 microns, 1 mm, 1.5 mm, 2 mm, 2.5 mm, 3 mm, 3.5 mm, 4 mm, 4.5mm, 5 mm, or greater. As seraphinite is a relatively soft mineral, it can be crushed into granular pieces or fragments using a metal hammer, press, or other metal tool.

As shown in FIGS. 1-8, the composition is formed into a disk 10 having a shape and dimensions that make it easy to place over an acupressure point. As used herein, a preferred form of the composite is referred to as a disk. The benefit of a disk-shape is that it provides an enhanced surface area relative to the size of the disk. Depending on the shape and vernacular, the formed piece may also be referred to as a button, stone, pellet, gem, pearl, blob, dome, marble, lens or other word.

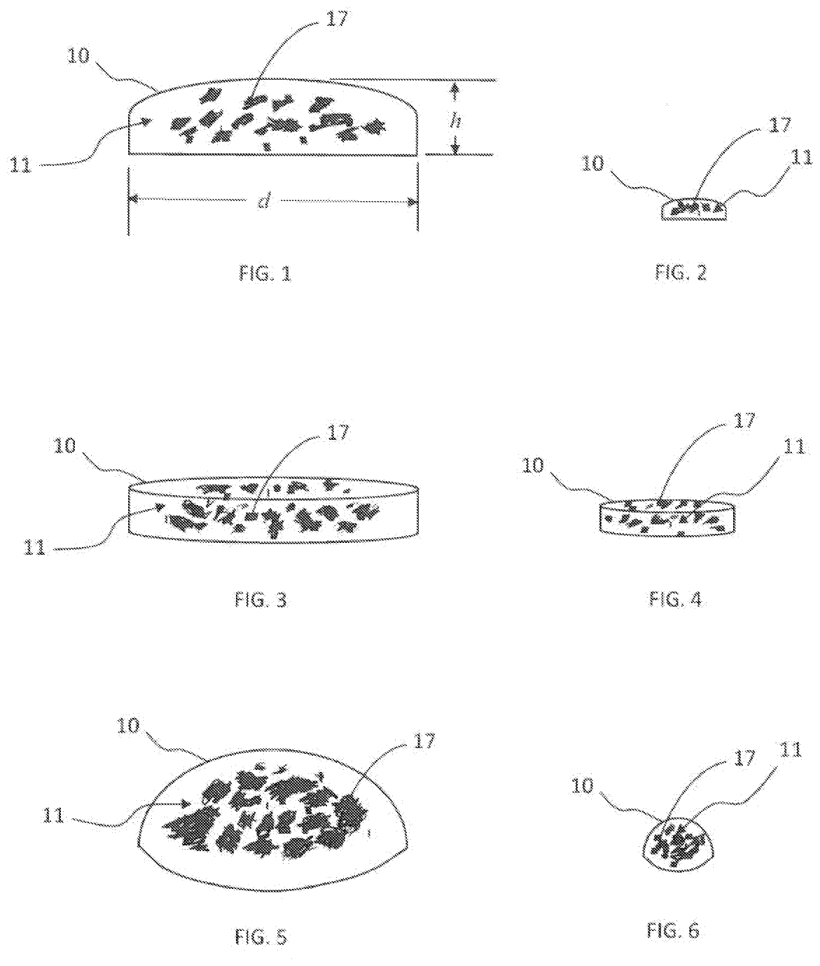

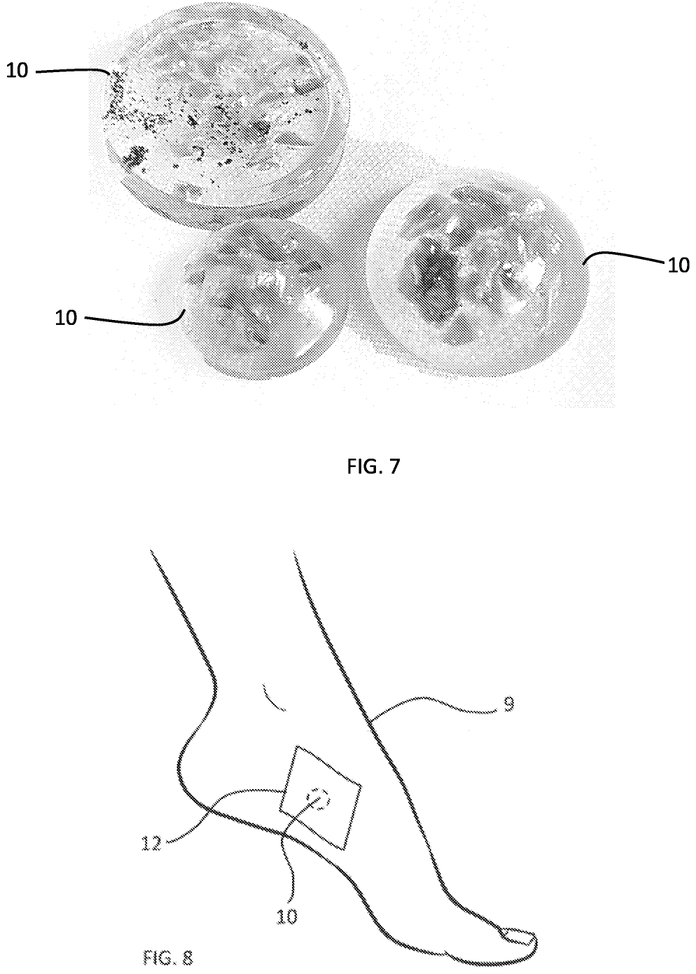

Typically the disks 10 are round with at least one flat side that aids in ensuring good contact between the disk and the person's skin, although they may take on other shapes including spheroidal, oval, polygonal, or a non-symmetric organic shape. FIGS. 1-2 show a round disk 10 with one flat side and a loaf-shaped top. FIGS. 3-4 show a round disk 10 with two flat sides. FIGS. 5-6 show a round disk 10 with one flat side and a dome-shaped top. In an alternative embodiment, the disks do not have a flat side. The diameter d ranges about 3 mm-40 mm, including 4 mm, 5 mm, 6 mm, 7 mm, 8 mm, 9 mm, 10 mm, 11 mm, 12 mm, 15 mm, 20 mm, 25 mm, 30 mm, and 35 mm, and the height h ranges from about 2 mm-10 mm, including 3 mm, 4 mm, 5 mm, 6 mm, 7 mm, 8 mm, and 9 mm. Any range encompassing these values are contemplated for the dimensions of the disk. Examples of various sizes and shapes of disks 10 are also shown in FIG. 7.

To make a disk, a portion of crushed seraphinite is placed into a mold having a desired shape, such as disk-shaped. Silicone molds work well, but the molds may be made of other materials such as plastic or metal. The mold can be of other shapes besides disk-shaped in other embodiments, including dome-shaped, pancake-shaped, loaf-shaped, tube-shaped, oval-shaped, sphere-shaped, cube-shaped, pyramid-shaped, diamond-shaped, or other geometric shape. A resin is poured into the mold in a liquid or viscous state and mixed with the crushed clinochlore 11 (individual flakes called out at 17) to distribute it through the disk or other shape. The resin eventually converts into rigid polymers that harden the composition. Natural resin, such as amber, balsam, or other plant exudates, or synthetic resin, such as acrylic or epoxy resin, may be used. Other examples of synthetic resins that may be used include polyester, phenolic, alkyd, polycarbonate, polyamide, polyurethane, silicone, polyethylene, polystyrene, and polypropylene. In some embodiments the resin is food-grade. Typically the resin is a two-part epoxy (i.e., monomeric resin and hardening agent). The resin may be translucent or transparent, in any color. Preferably the resin is clear with no color to allow the beauty of the crushed seraphinite to be seen. The crushed clinochlore can be present in the composition in the range of 10% to 90% by volume, including 10%-20%, 20%-30%, 30%-40%, 40%-50%, 50%-60%, 60%-70%, 70%-80%, 80%-90%, 40%-60%, 35%-65%, and 45%-55%.

The resin is allowed to cure. As the resin hardens, it contracts against the seraphinite, fragments by stressing and slightly deforming them. Owing to its piezo crystalline characteristic, as the seraphinite is mechanically stressed, a voltage potential difference across its opposite faces is created. The hardened resin creates a lasting stress on the crystal. In practice, the stressed crystal becomes a kind of tiny battery with a positive charge on one face and a negative charge on the opposite face. A mild current flows when the two faces are connected together at the skin to make a circuit. The composite is placed over an acupressure or acupuncture point on a person's body and secured to the skin with medical grade removable adhesive tape. Typically the disk or other composite form is left in place until the person senses relaxation, which can be just a few minutes or for hours at a time. Each formed composite can be sanitized and re-used.

Multiple disks or other forms of the composite can be worn at different points on a person at the same time. The formed composites can be used by massage therapists, energy workers, chiropractors and acupuncturists to stimulate acupressure/acupuncture points and trigger points. The composites provide for a convenient application of a piezoelectric effect of the crystals/mineral to areas of the body, and in particular acupressure or acupuncture meridian points. Three hundred sixty one classical acupuncture points have been named and published by the World Health Organization (see Regional Office for the Western Pacific, WHO. 1991. A Proposed Standard International Acupuncture Nomenclature Report of a WHO Scientific Group; and Regional Office for the Western Pacific, WHO. 1993. Standard Acupuncture Nomenclature, 2nd ed. World Health Organization; both incorporated by reference in their entireties). These acupressure or acupuncture points are located along twenty major cutaneous and subcutaneous meridians, including twelve primary medians (Lung, Large Intestine, Stomach, Spleen, Heart, Small Intestine, Bladder, Kidney, Pericardium, Triple Energizer, Gallbladder, and Liver) and eight extraordinary meridians (Governing Vessel, Conception Vessel, Thrusting Vessel, Belt Vessel, Yin Heel Vessel, Yang Heel Vessel, Yin Link Vessel, and Yang Link Vessel). The mild stimulus of points along these meridians can relax a person treated with the formed composites. The composite disks (or other formed shapes) can be worn for a short duration or longer duration and also while the user is resting or asleep. The composites can be applied for 1-5 minutes, 10 minutes, 15 minutes, 20 minutes, 30 minutes, 45 minutes, 1 hour, 2 hours, 3 hours, 4 hours, 5 hours, 6 hours, 8 hours, 10 hours, 12 hours, 24 hours, 48 hours, 72 hours, 4 days, 5 days, 6 days, 7 days, 2 weeks, 3 weeks, 4 weeks, 2 months, 3 months, or longer or any time desired by the user. According to one embodiment, a method of treating a user includes applying a composition comprising a piezoelectric material and a polymerized resin to an acupressure or acupuncture point of a user in need of treatment. FIG. 8 illustrates an example of the method in practice, where a disk 10 of the composite is applied to an acupuncture point on a user's foot 9 through adhesive tape 12. The method of relaxation has the advantage of stimulating energy flow in the body of the user without the need for a practitioner to insert needles or constantly apply pressure.

In other embodiments, other piezoelectric crystals such as langasite, lithium niobate, lithium tantalate, quartz, berlinite, and lead titanate can be substituted in lieu of clinochlore in the composition and in treatment of the user.

The present disclosure has described particular implementations having various features. In light of the disclosure provided above, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A composition comprising at least one clinochlore and a resin.

2. The composition of claim 1, wherein the resin is in polymerized form.

3. The composition of claim 1, wherein the at least one clinochlore is seraphinite.

4. The composition of claim 3, further comprising at least one additional clinochlore chosen from colerainite, kämmerite, pennine, penninite, prochlorite, ripidolite, and sheridanite.

5. The composition of claim 1, wherein the resin is a plant resin.

6. The composition of claim 5, wherein the resin is amber.

7. The composition of claim 1, wherein the resin is synthetic.

8. The composition of claim 7, wherein the resin is acrylic.

9. The composition of claim 7, wherein the resin is epoxy.

10. The composition of claim 7, wherein the resin is polyester, phenolic, alkyd, polycarbonate, polyamide, polyurethane, silicone, polyethylene, polystyrene, or polypropylene.

11. The composition of claim 2, wherein the composition is formed into a disk.

12. The composition of claim 11, wherein the disk has a diameter in the range of 3 mm to 40 mm.

13. The composition of claim 11, wherein the disk has a height in the range of 2 mm to 10 mm.

14. The composition of claim 1, wherein the at least one clinochlore comprises clinochlore fragments.

15. The composition of claim 2, wherein the at least one clinochlore comprises clinochlore fragments.

16. The composition of claim 15, wherein the clinochlore fragments are piezoelectric, and the polymerized resin mechanically stresses the clinochlore fragments to produce a voltage potential.

17. A method of relaxation for a user, comprising applying a composition comprising a piezoelectric material and a polymerized resin to at least one acupuncture or acupressure point of a user.

18. The method of claim 17, wherein the piezoelectric material is a clinochlore.

19. The method of claim 18, wherein the clinochlore is seraphinite.

20. The method of claim 17, wherein the composition is applied to the at least one acupuncture or acupressure point through adhesive tape.
